(19) United States
(12) Patent Application Publication
(10) Pub. No.: US 2023/0302173 A1
(43) Pub. Date: Sep. 28, 2023

(54) ULTRAVIOLET LIGHT IRRADIATION SYSTEM AND ULTRAVIOLET LIGHT IRRADIATION METHOD

(71) Applicant: NIPPON TELEGRAPH AND TELEPHONE CORPORATION, Tokyo (JP)

(72) Inventors: Tomohiro TANIGUCHI, Musashino-shi, Tokyo (JP); Ayako IWAKI, Musashino-shi, Tokyo (JP); Kazuhide NAKAJIMA, Musashino-shi, Tokyo (JP); Nobutomo HANZAWA, Musashino-shi, Tokyo (JP); Takashi MATSUI, Musashino-shi, Tokyo (JP); Yuto SAGAE, Musashino-shi, Tokyo (JP); Chisato FUKAI, Musashino-shi, Tokyo (JP)

(73) Assignee: NIPPON TELEGRAPH AND TELEPHONE CORPORATION, Tokyo (JP)

(21) Appl. No.: 18/032,495

(22) PCT Filed: Oct. 21, 2020
(86) PCT No.: PCT/JP2020/039580

§ 371 Date: Apr. 18, 2023

(51) Int. Cl. A61L 2/10; A61L 2/24

(52) U.S. Cl. CPC A61L 2/10; A61L 2/24; A61L 2202/11; G02B 6/4206

(57) ABSTRACT

In order to solve the above problems, an object of the present invention is to provide an ultraviolet light irradiation system and an ultraviolet light irradiation method with a few transmission losses in an optical fiber.

The ultraviolet light irradiation system 301 includes: a light source unit 11 for injecting transmission infrared light into an optical fiber 50, a wavelength conversion unit 12 for converting the transmission infrared light propagated through the optical fiber 50 into ultraviolet light, and an irradiation unit 13 for irradiating the ultraviolet light on a desired place. The light source unit 11 has a polarization scrambler 11b, and infrared light output from one infrared light source 11a is made to be transmission infrared light by the polarization scrambler 11b.

### TECHNICAL FIELD

The present disclosure relates to an ultraviolet light irradiation system in which a light source and an ultraviolet light irradiation unit are separated from each other, and an ultraviolet light irradiation method thereof.

### BACKGROUND ART

For the purpose of preventing infectious diseases, there is an increasing demand for systems for sterilization and inactivation of viruses using ultraviolet light. Such systems have three major categories of products. In the following description, sterilization and inactivation of viruses is described as “sterilization or the like”.

(1) Movement type sterilization robot (see, for example, NPL 1)

A movement type sterilization robot is an autonomous movement type robot for radiating ultraviolet light. The robot emits ultraviolet light while moving in a room in a building such as a hospital room, thereby automatically achieving sterilization or the like over a wide range without human intervention.

(2) Stationary air purifier (see, for example, NPL 2)

A stationary air purifier is a device that is installed on a ceiling or in a predetermined place in a room and performs sterilization or the like while circulating the air in the room. Since the device does not directly emit ultraviolet light and does not affect the human body, highly safe sterilization or the like can be performed.

(3) Portable sterilization device (see, for example, NPL 3) A portable sterilization device is a portable type device mounted with an ultraviolet light source. A user can use the device in various places by carrying the device to an area of an object such as sterilization and irradiating the object such as sterilization with ultraviolet light.

The above-mentioned systems have the following problems.

(1) Movement type sterilization robot

Since the sterilization robot emits high-output ultraviolet light, the device is large-scale and expensive, and there is a problem that it is difficult to realize an economical system.

(2) Stationary air purifier

There is a problem that the stationary air purifier cannot directly emit ultraviolet light to a place to be sterilized because of a method of sterilizing circulated indoor air.

(3) Portable sterilization device

Since the user may not necessarily have the skills or knowledge, there is a risk of the human body being affected depending on the method of use, and there is a problem that the user may not know whether the operation has been performed so that a sufficient sterilization effect has been obtained at the target location.

In other words, there has been a demand for a system having high economic efficiency, which can directly irradiate a desired portion with ultraviolet light regardless of the presence or absence of the skill of the user. As such a system, a system for transmitting ultraviolet light through an optical fiber is considered. By transmitting the ultraviolet light from the light source by using the thin and easily bendable optical fiber, flexibility is obtained that the ultraviolet light output from the tip of the optical fiber can be irradiated to a place to be sterilized or the like at a pinpoint. Further, by using a Point-to-Multi-Point (P-MP) configuration such as an FTTH (Fiber To The Home) of an optical communication system, the light from a single light source is shared at a plurality of positions to achieve economy.

### CITATION LIST

### Non Patent Literature

[NPL 1] Kantum Ushikata Co., Ltd website, https://www.kantum.co.jp/product/haizen robot/Deep-Blue penguinn robot/UVD robot
[NPL 2] Iwasaki Electric Industry Co., Ltd. website, https://www.iwasaki.co.jp/optics/sterilization/air/air03.html
[NPL 3] Funakoshi Co., Ltd. website, https://www.funakoshi.co.jp/contents/68182
[NPL 4] Fujikura Ltd. website,
https://www.fujikura.co.jp/products/optical/appliedoptics/02/2 051999_11309.html
[NPL 5] Mitsubishi Cable Industries, Ltd. website, https://www.mitsubishi-cable.co.jp/ja/products/group/optical-fiber/large.html
[NPL 6] S. Takahasi, and others, “Wavelength Conversion Technology utilizing Linearly-Polarized Fiber Laser and its Application”, Fujikura Technical Journal, Vol. No. 126, pp. 25-30, July 2014.

### SUMMARY OF INVENTION

### Technical Problem

However, in the ultraviolet irradiation system using optical fibers, there is a problem that long-distance transmission is difficult due to the influence of fiber transmission characteristics in the ultraviolet region. For example, there are reports about a large-diameter fiber in which a core is doped with an OH group (for example, NPL 4, 5 reference), and a hollow optical fiber (for example, NPL 6 reference). According to the reports, in a wavelength region of 260 nm to 280 nm effective for sterilization or the like, a transmission loss of about 0.3 dB/m is generated. This transmission loss is 1500 times the transmission loss in the communication wavelength band (0.0002 dB/m, 1.5 um band), and the long-distance transmission of ultraviolet light is non-realistic.

Therefore, the present invention is intended to provide an ultraviolet light irradiation system and a ultraviolet light irradiation with a few transmission losses in the optical fiber to solve the above problems.

### Solution to Problem

In order to achieve the above object, the ultraviolet light irradiation system according to the present invention transmits light in an infrared region which can be transmitted through an optical fiber with a low loss, and converts the wavelength of light received on the light irradiation side into the ultraviolet region by a nonlinear optical effect.

Specifically, a first ultraviolet light irradiation system according to the present invention includes:
a light source unit that injects transmission infrared light of two polarized waves orthogonal to each other into an optical fiber,
a wavelength conversion unit that converts the transmission infrared light propagated through the optical fiber into ultraviolet light, and
an irradiation unit that irradiates the ultraviolet light to a desired location.

Further, a first ultraviolet light irradiation method according to the present invention performs:
injecting transmission infrared light of two polarized waves orthogonal to each other into an optical fiber,
converting the transmission infrared light propagated through the optical fiber into ultraviolet light, and
irradiating the ultraviolet light to a desired location.

High wavelength conversion efficiency can be obtained by using a quasi-phase matching (QPM) wavelength conversion element such as PPLN (Periodically Poled LiNbO3) as a wavelength conversion unit having a nonlinear optical effect. According to this configuration, as described above, it is possible to realize an ultraviolet light irradiation system that has the flexibility to irradiate ultraviolet light with optical fibers to pinpoint locations where sterilization or the like is desired, as well as the economic efficiency of sharing light sources and solving the problem of transmission loss of ultraviolet light by optical fibers, enabling long distance transmission.

Note that the QPM wavelength conversion element has polarization dependency. Since the polarization of light usually fluctuates during transmission in optical fibers, it is difficult to maintain a specific state of polarization at the point of input to the wavelength conversion element at the light irradiation side. Therefore, the power of the ultraviolet light generated by wavelength conversion is fluctuated by the polarization fluctuation during the transmission of the optical fiber, and the stable effect of sterilization or the like is hardly obtained.

With respect to the polarization fluctuation during the transmission of the optical fiber, the ultraviolet irradiation system transmits the Infrared light through the optical fiber by two polarized waves orthogonal to each other. Even if the polarized wave is fluctuated by transmission, it is possible by two polarized waves that the wavelength of either polarized wave can be converted with high efficiency by the wavelength conversion element, so that the power of the ultraviolet light generated by the wavelength conversion is stabilized. Therefore, the ultraviolet light irradiation system can obtain stable effects such as sterilization or the like.

Accordingly, the present invention can provide an ultraviolet light irradiation system and an ultraviolet light irradiation method with a few transmission losses in an optical fiber.

The specific configuration of injecting infrared light of two polarized waves orthogonal to each other to the optical fiber is as follows.

In the first configuration, the light source unit has a polarization scrambler, and infrared light output from one infrared light source is used as the transmission infrared light by the polarization scrambler. In other words, the light source unit performs polarization scramble processing on the transmission infrared light.

In the second configuration, the light source unit has a polarization multiplexer, and infrared light output from two infrared light sources having different output wavelengths is multiplexed by the polarization multiplexer to obtain the transmission infrared light. In other words, two-wavelength Infrared light is generated, and the Infrared light is multiplexed in a state in which the polarized waves are orthogonal to each other, and then transmitted from the optical fiber.

Further, the configuration may be as follows.

A second ultraviolet light irradiation system according to the present invention includes:
a light source unit that injects transmission infrared light into an optical fiber,
a wavelength conversion unit that converts the transmission infrared light propagated through the optical fiber into ultraviolet light, and
an irradiation unit that irradiates the ultraviolet light to a desired location; wherein
the wavelength conversion unit has a polarization diversity configuration for separating the transmission infrared light into orthogonal polarized waves and converting each of the transmission infrared light into ultraviolet light.

Further, a second ultraviolet light irradiation method according to the present invention performs:
injecting transmission infrared light into an optical fiber, separating the transmission infrared light propagating through the optical fiber into orthogonal polarized waves,
converting each of the separated transmission infrared light into ultraviolet light, and
irradiating the ultraviolet light to a desired location.

Since this configuration also transmits infrared light with the optical fiber, as the first ultraviolet light irradiation system, it is possible to realize an ultraviolet light irradiation system that has the flexibility to irradiate ultraviolet light with optical fibers to pinpoint locations where sterilization or the like is desired, as well as the economic efficiency of sharing light sources and solving the problem of transmission loss of ultraviolet light by optical fibers, enabling long distance transmission.

Further, since the wavelength conversion unit on the light irradiation side is configured to have a polarization diversity configuration with respect to polarization fluctuation during optical fiber transmission, even if polarization fluctuates during optical fiber transmission, wavelength conversion can be performed with high efficiency by any of the wavelength conversion elements, the power of the ultraviolet light generated by wavelength conversion is stabilized. Therefore, the ultraviolet light irradiation system also provides stable effects such as sterilization or the like.

Accordingly, the present invention can provide an ultraviolet light irradiation system and an ultraviolet light irradiation method with a few transmission losses in an optical fiber.

The wavelength conversion unit of the ultraviolet light irradiation system is integrated with the irradiation unit, and the ultraviolet light may be directly supplied to the irradiation unit. In the ultraviolet light irradiation system, the wavelength conversion unit and the irradiation unit are physically separated, and the wavelength conversion unit may supply the ultraviolet light to the irradiation unit via an optical fiber.

Further, it is preferred that, at least one of the optical fibers that propagates the transmission infrared light and the optical fiber that supplies the ultraviolet light from the wavelength conversion unit to the irradiation unit is, any one of a solid core optical fiber, a hole-assisted optical fiber, a hole-structured optical fiber, a hollow core optical fiber, a coupled core type optical fiber, a solid core type multi-core optical fiber, a hole-assisted type multi-core optical fiber, a hole-structured type multi-core optical fiber, a hollow core type multi-core optical fiber, and a coupled core type multi-core optical fiber.

It should be noted that the inventions described above can be combined as much as possible.

### Advantageous Effects of Invention

The present invention can provide an ultraviolet light irradiation system and an ultraviolet light irradiation method with a few transmission losses in an optical fiber. Further, the present invention can provide an ultraviolet light irradiation system and an ultraviolet light irradiation method that output ultraviolet light of stable power without depending on polarization fluctuation occurring during optical fiber transmission.

### BRIEF DESCRIPTION OF DRAWINGS

FIG. 1 is a drawing for explaining the ultraviolet light irradiation system according to the present invention.

FIG. 2 is a drawing for explaining the light source unit of the ultraviolet light irradiation system according to the present invention.

FIG. 3 is a drawing for explaining the light source unit of the ultraviolet light irradiation system according to the present invention.

FIG. 4 is a drawing for explaining the wavelength conversion unit of the ultraviolet light irradiation system according to the present invention.

FIG. 5 is a drawing for explaining the wavelength conversion unit of the ultraviolet light irradiation system according to the present invention.

FIG. 6 is a drawing for explaining the ultraviolet light irradiation system according to the present invention.

FIG. 7 is a drawing for explaining the ultraviolet light irradiation system according to the present invention.

FIG. 8 is a drawing for explaining the ultraviolet light irradiation system according to the present invention.

FIG. 9 is a drawing for explaining a cross section of an optical fiber.

FIG. 10 is a drawing for explaining the ultraviolet light irradiation method according to the present invention.

FIG. 11 is a drawing for explaining the ultraviolet light irradiation method according to the present invention.

### DESCRIPTION OF EMBODIMENTS

Embodiments of the present invention will be described with reference to the accompanying drawings. The embodiments described below are examples of the present invention and the present invention is not limited to the following embodiments. Note that constituent elements with the same reference signs in the present description and the drawings shall indicate the same thing to each other.

### Embodiment 1

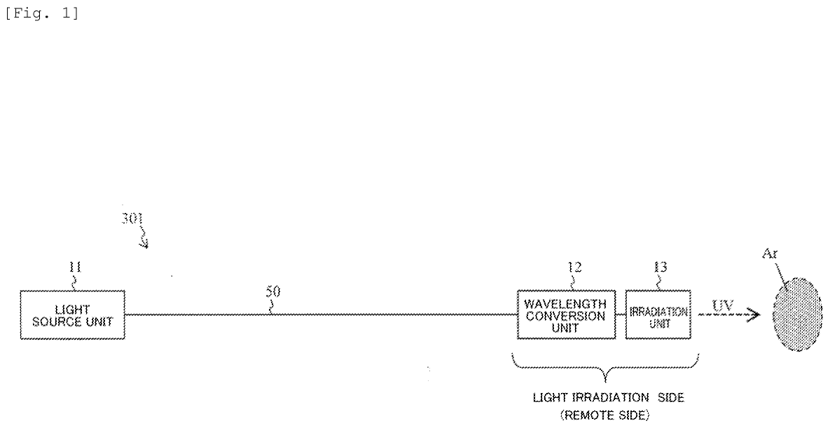

FIG. 1 is a diagram for explaining the ultraviolet light irradiation system 301 of the present embodiment. The ultraviolet light irradiation system 301 includes the light source unit 11 for injecting the transmission infrared light into the optical fiber 50, the wavelength conversion unit 12 for converting the transmission infrared light propagated through the optical fiber 50 into ultraviolet light, the irradiation unit 13 that irradiate the ultraviolet light to a desired location.

The light source unit 11 has a light source in an infrared region such as 1064 nm, for example, and injects output light from the light source into the optical fiber 50. In the present specification, the output light from the light source is referred to as “infrared light”, and the light transmitted through the optical fiber 50 is referred to as “transmission infrared light”.

The wavelength conversion unit 12 has a wavelength conversion device with such as a nonlinear optical crystal. The wavelength conversion unit 12 generates ultraviolet light by performing wavelength conversion of, for example, four-fold wave generation and five-fold wave generation to the transmission infrared light in the infrared region transmitted by the optical fiber 50.

The irradiation unit 13 irradiates the ultraviolet light output from the wavelength conversion unit 12 to a desired object area Ar of sterilization or the like with. The irradiation units 13 are constituted of an optical system such as a lens designed for wavelengths in the ultraviolet region. In this embodiment, the wavelength conversion unit 12 and the irradiation unit 13 are integrated.

Since the ultraviolet light irradiation system 301 transmits the infrared light having a small transmission loss with the optical fiber 50, the problem of the transmission loss of the ultraviolet light by the optical fiber is solved and long-distance transmission is made possible.

### Embodiment 2

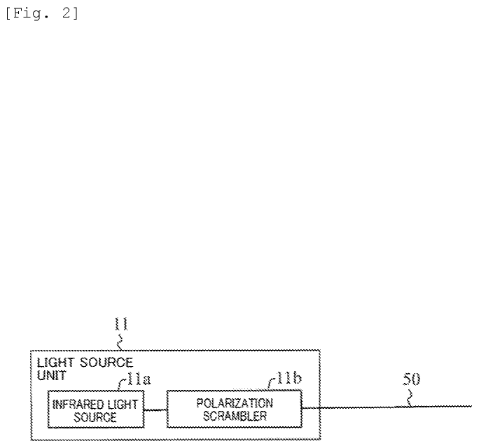

In the ultraviolet light irradiation system 301 described with reference to FIG. 1, it is preferable that the transmission infrared light transmitted through the optical fiber 50 is transmitted by two polarized waves orthogonal to each other. FIG. 2 is a view for explaining the light source unit 11 for outputting transmission Infrared light of two polarized waves orthogonal to each other. The light source unit 11 has a polarization scrambler 11b, and infrared light outputted from one infrared light source 11a is made to be transmission infrared light by the polarization scrambler 11b.

The light source unit 11 shown in FIG. 2 is configured to polarize and scrambler infrared light of a single wavelength. The infrared light source 11a outputs infrared light of a wavelength at which wavelength conversion is performed with high efficiency by the wavelength conversion unit 12. The polarization scrambler 11b is configured to switch the polarization state of the infrared light from the infrared light source 11a to a state orthogonal to each other at a fixed period by using a polarization controller, or to switch the polarization state at a fixed period by a polarization modulator using lithium niobate (LN) or the like.

The structure of the light source unit 11 as shown in FIG. 2 can be constituted of a single light source, and the structure can be simplified.

### Embodiment 3

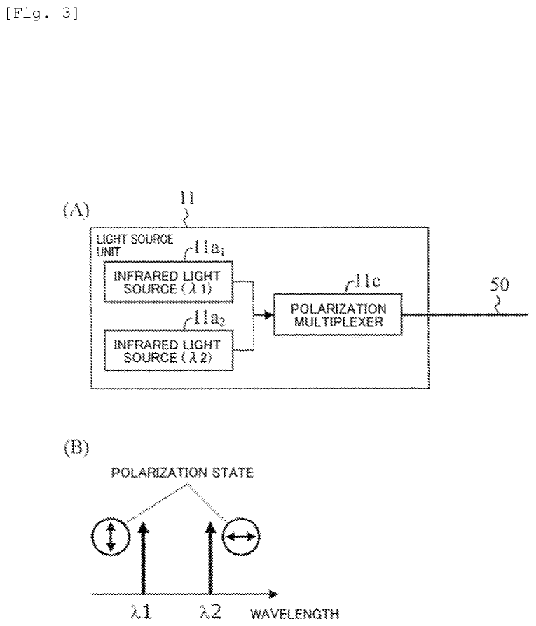

In the ultraviolet light irradiation system 301 described with reference to FIG. 1, it is preferable that the transmission infrared light transmitted through the optical fiber 50 is transmitted by two polarized waves orthogonal to each other. FIG. 3(A) is a drawing for explaining the light source unit 11 for outputting transmission infrared light of two polarized waves orthogonal to each other. The light source unit 11 has a polarization multiplexer 11c, and is characterized in that infrared light outputted from two infrared light sources (11a₁, 11a₂) having different output wavelengths are multiplexed by the polarization multiplexer 11c to obtain the transmission infrared light.

The light source unit 11 shown in FIG. 3(A) is configured to perform orthogonal polarization synthesis of Infrared light having two wavelengths. Both of the two infrared light sources (11a₁, 11a₂) output infrared light of a wavelength at which wavelength conversion is performed with high efficiency by the wavelength conversion unit 12. As shown in FIG. 3(B), the polarization multiplexer 11c multiplexes the infrared light of the wavelength λ1 from the infrared light source 11a₁ and the infrared light of the wavelength λ2 from the infrared light source 11a₂ so that the polarization states are orthogonal to each other, and outputs the multiplexed light to an optical fiber 50.

By structuring the light source unit 11 to use two light sources as shown in FIG. 3(A), the optical power transmitted to the optical fiber 50 can be increased, and as a result, the power of the ultraviolet light output from the wavelength conversion unit 12 can be increased.

### Embodiment 4

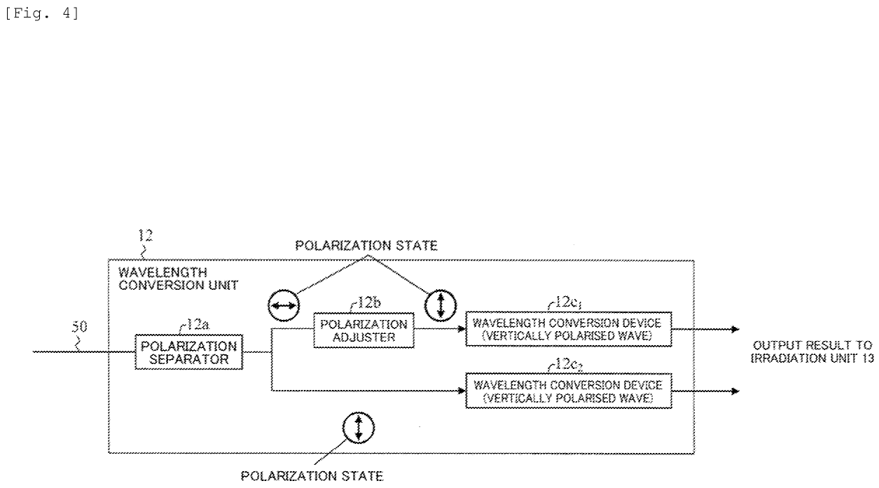

In the ultraviolet light irradiation system 301 described with reference to FIG. 1, it is preferable that the wavelength conversion unit 12 has a polarization diversity configuration using two wavelength conversion devices having orthogonal polarization directions for obtaining high efficiency. FIG. 4 is a diagram for explaining the wavelength conversion unit 12 having a polarization diversity configuration. The wavelength conversion unit 12 has a polarization diversity configuration in which the transmission infrared light transmitted by the optical fiber 50 is separated into orthogonal polarized waves and each transmission infrared light is converted into ultraviolet light.

The wavelength conversion unit 12 shown in FIG. 4 rotates one polarized wave. A polarization separator 12a separates the transmission infrared light from the optical fiber 50 into a vertically polarized wave component and a horizontally polarized wave component and outputs the separated lights. A polarization adjuster 12b adjusts one of the lights (in FIG. 4, the horizontally polarized wave) to the other polarization wave (in FIG. 4, the vertically polarized wave). The wavelength conversion devices (12c₁, 12c₂) are non-linear optical crystals having polarization dependency, and infrared light of one polarization (in FIG. 4, the vertically polarized wave) can be converted into ultraviolet light with high efficiency. In the example shown in FIG. 4, a wavelength conversion unit 12 directly inputs infrared light of vertical polarization separated by a polarization separator 12a to a wavelength conversion device 12c₂, converts infrared light of horizontal polarization into infrared light of vertical polarization by a polarization adjuster 12b, and then inputs it to a wavelength conversion device 12c₁.

Since by making the wavelength conversion unit 12 into the structure shown in FIG. 4, two wavelength conversion devices can be made into a single type, parts procurement and management can be facilitated.

### Embodiment 5

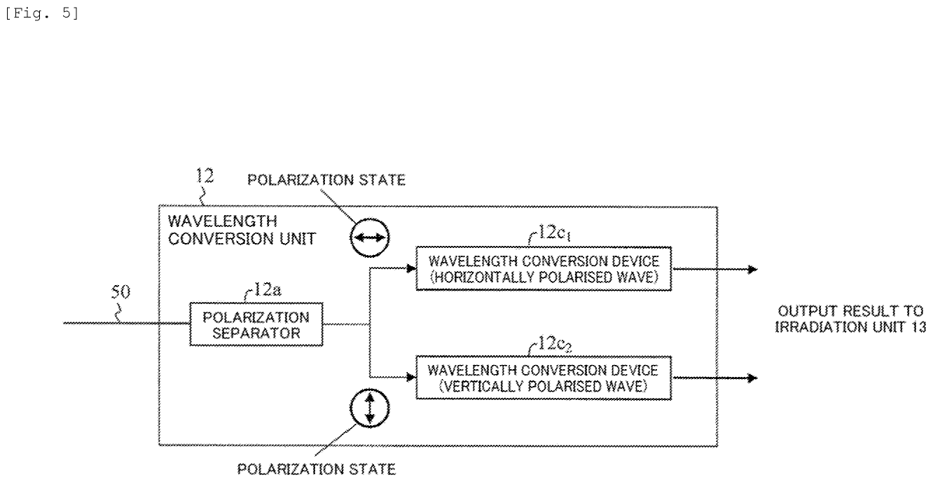

FIG. 5 is a diagram for explaining a wavelength conversion unit 12 having another polarization diversity configuration. The wavelength conversion unit 12 shown in FIG. 5 is provided with wavelength conversion devices adapted to polarized waves. A polarization separator 12a separates the transmission infrared light from the optical fiber 50 into a vertically polarized wave component and a horizontally polarized wave component and outputs the separated lights. The wavelength conversion devices (12c₁, 12c₂) are nonlinear optical crystals having polarization dependency, and can convert infrared light of one polarization into ultraviolet light with high efficiency (in FIG. 5, the wavelength conversion device 12c₁ can convert infrared light of horizontal polarization, and the wavelength conversion device 12c₂ can convert light of vertical polarization). In the example shown in FIG. 5, the wavelength conversion unit 12 inputs directly the infrared light of the vertically polarized wave separated by the polarization separator 12a to the wavelength conversion device 12c₂, and inputs directly the infrared light of the horizontally polarized wave to the wavelength conversion device 12c₁.

By making the wavelength conversion unit 12 into the structure shown in FIG. 5, a polarization adjuster 12b is not required and the number of components can be reduced as compared with the structure shown in FIG. 4.

### Embodiment 6

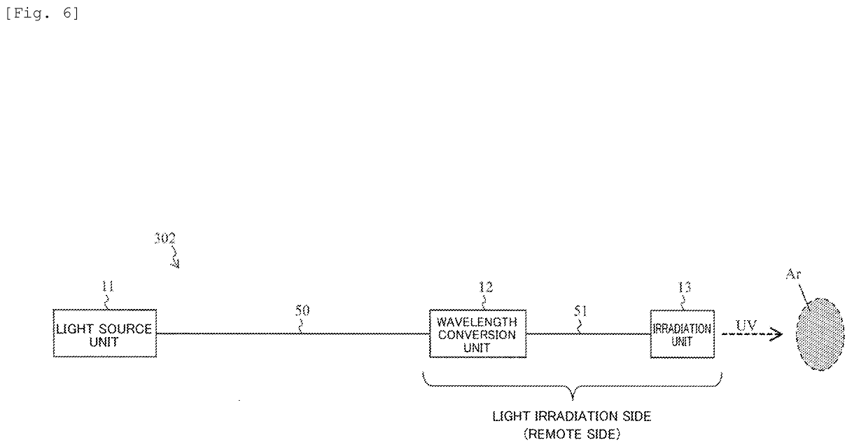

FIG. 6 is a diagram illustrating an ultraviolet light irradiation system 302 according to the present embodiment. The ultraviolet light irradiation system 302 differs from the ultraviolet light irradiation system 301 shown in FIG. 1 in that the wavelength conversion unit 12 and the irradiation unit 13 are separated from each other, and they are connected to each other by an optical fiber 51.

The optical fiber 51 is an optical fiber for transmitting ultraviolet light, and transmits the ultraviolet light output from the wavelength conversion unit 12 to the irradiation unit 13. The optical fiber 51 is, for example, a large-diameter optical fiber in which an OH group is doped in a core or a hollow optical fiber in which a portion for guiding light is a cavity.

Since the ultraviolet light irradiation system 302 does not need to install the wavelength conversion unit 12 near the irradiation unit 13, it is possible to more flexibly irradiate a finer place than the ultraviolet light irradiation system 301 shown in FIG. 1 with ultraviolet light.

### Embodiment 7

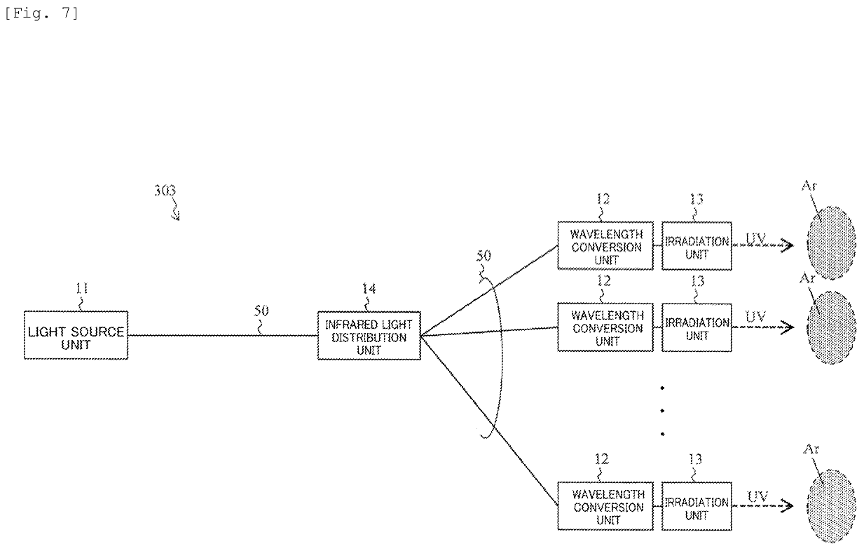

FIG. 7 is a diagram illustrating an ultraviolet light irradiation system 303 according to the present embodiment. The ultraviolet light irradiation system 303 differs from the ultraviolet light irradiation system 301 shown in FIG. 1 in that a plurality of wavelength conversion units 12 and irradiation units 13 are provided, and an infrared light distribution unit 14 is provided in the middle of an optical fiber 50.

An infrared light distribution unit 14 distributes the transmission infrared light from the light source unit 11 to a plurality of outputs by using an optical splitter such as a PLC type or a fiber type. The distributed transmission infrared light is input to each wavelength conversion unit 12.

Since the ultraviolet light irradiation system 303 shares a single light source unit 11 by the plurality of irradiation units 13, the system can be constructed with time. Further, since the ultraviolet light irradiation system 303 transmits light from the light source unit 11 to the wavelength conversion unit 12 (the irradiation unit 13 integrated with the wavelength conversion unit 12) in an infrared region where transmission loss in the optical fiber is low, the transmission distance between the infrared light distribution unit 14 and the irradiation unit 13 can be extended, and flexible system design can be performed.

### Embodiment 8

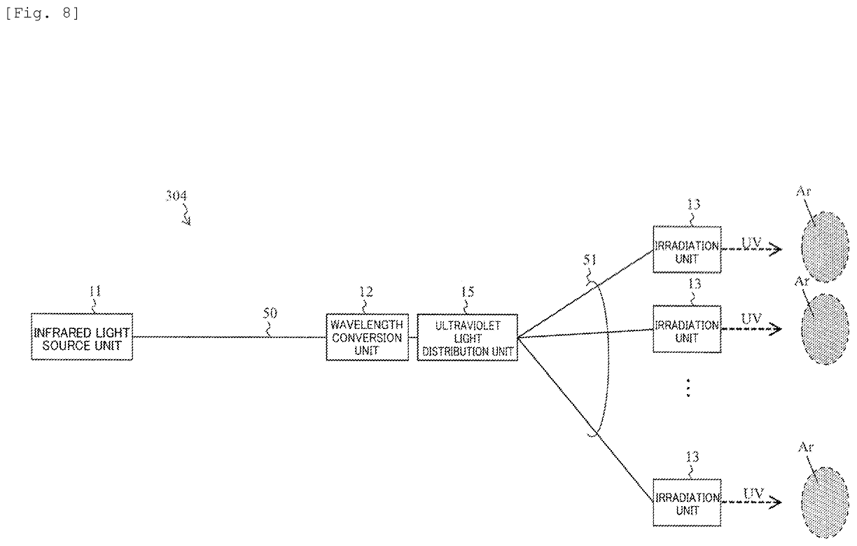

FIG. 8 is a diagram illustrating an ultraviolet light irradiation system 304 according to the present embodiment. The ultraviolet light irradiation system 304 differs from the ultraviolet light irradiation system 302 shown in FIG. 6 in that a plurality of irradiation units 13 are provided and an ultraviolet light distribution unit 15 is provided at the subsequent stage of the wavelength conversion unit 12.

An ultraviolet light distribution unit 15 distributes the ultraviolet light from the wavelength conversion unit 12 to a plurality of outputs by using an optical splitter such as a PLC type or a fiber type. The distributed ultraviolet light is input to each irradiation unit 13.

Since the ultraviolet light irradiation system 304 shares the single light source unit 11 and the wavelength conversion section 12 by the plurality of irradiation units 13, the system can be constructed more economically than the ultraviolet light irradiation system 303 shown in FIG. 7. In particular, the ultraviolet light irradiation system 304 is effective in such a system configuration that the transmission distance between the ultraviolet light distribution unit 12 and the irradiation section 13 is not so long and the transmission loss of ultraviolet light does not become a problem.

### Embodiment 9

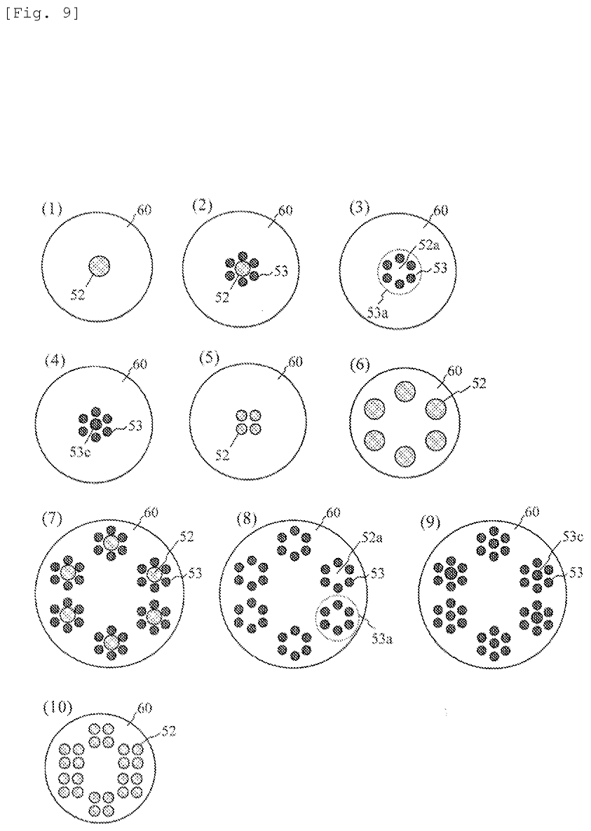

The optical fiber having a cross-sectional structure as illustrated in FIG. 9 can be used as the optical fiber (50, 51), in the described above ultraviolet light irradiation system (301 to 304).

(1) Solid Core Optical Fiber

The optical fiber has one solid core 52 having a refractive index higher than that of a clad 60 in the clad 60. “Solid” means “not hollow.” The solid core can also be realized by forming an annular low refractive index region in the clad.

(2) Hole-Assisted Optical Fiber

The optical fiber has a solid core 52 and a plurality of holes 53 disposed on the outer periphery of the solid core 52 in a clad 60. The medium of the hole 53 is air, and the refractive index of the air is sufficiently smaller than that of quartz glass. Therefore, the hole-assisted optical fiber has a function of returning light leaking from the core 52 by bending or the like to the core 52 again, and has a characteristic of small bending loss.

(3) Hole-Structured Optical Fiber

The optical fiber has a hole group 53a of a plurality of holes 53 in a clad 60, and has a refractive index effectively lower than that of a host material (glass or the like). This structure is called a photonic crystal fiber. This structure can have a structure in which a high refractive index core having a changed refractive index is not present, and light can be confined by making a region 52a surrounded by the holes 53 an effective core region. As compared with an optical fiber having a solid core, the photonic crystal fiber can reduce the influence of absorption and scattering loss of the core by an additive, and can realize optical characteristics which cannot be realized by the solid optical fiber such as reduction of bending loss and control of nonlinear effect.

(4) Hollow Core Optical Fiber

The core region of the optical fiber is formed of air. Light can be confined in the core region by taking a photonic band gap structure by a plurality of holes or an anti-resonant structure by a glass thin wire in the clad region. The optical fiber has a small nonlinear effect and can supply a high output or high energy laser.

(5) Coupled Core Type Optical Fiber

In the optical fiber, a plurality of solid cores 52 having a high refractive index are disposed in close proximity to each other in a clad 60. The optical fiber guides light by light wave coupling between the solid cores 52. Since the coupled core type optical fiber can disperse and send light by the number of cores, there is an advantage that the power can be increased by that amount and efficient sterilization can be performed, or the deterioration of the fiber due to ultraviolet rays can be relaxed and the service life can be prolonged.

(6) Solid Core Type Multi-Core Optical Fiber

In the optical fiber, a plurality of solid cores 52 having a high refractive index are disposed apart from each other in a clad 60. The optical fiber guides light in a state where the influence of the light wave coupling can be ignored by sufficiently reducing the light wave coupling between the solid cores 52. Therefore, the solid core type multi-core optical fiber has an advantage that each core can be handled as an independent waveguide.

(7) Hole-Assisted Type Multi-Core Optical Fiber

The optical fiber has a structure in which a plurality of the hole structures and core regions of the above (2) are disposed in a clad 60.

(8) Hole-Structured Type Multi-Core Optical Fiber

The optical fiber has a structure in which a plurality of the hole structures of the above (3) are disposed in a clad 60.

(9) Hollow Core Type Multi-Core Optical Fiber

The optical fiber has a structure in which a plurality of the hole structures of the above (4) are disposed in a clad 60.

(10) Coupled Core Type Multi-Core Optical Fiber

The optical fiber has a structure in which a plurality of the coupled core structures of the above (5) are disposed in a clad 60.

(Ultraviolet Light Irradiation Method)

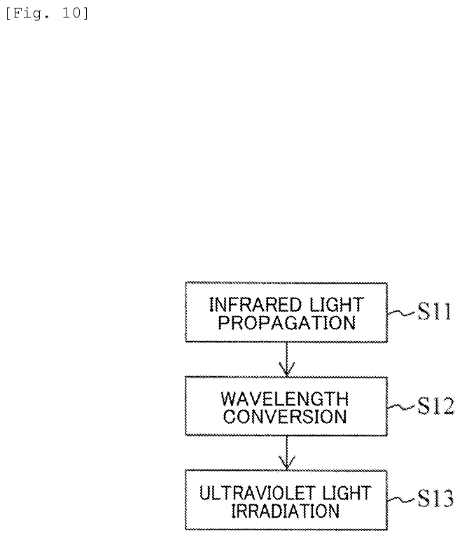

FIG. 10 is a flow chart for explaining an ultraviolet light irradiation method in the ultraviolet light irradiation system having the light source unit 11 shown in FIGS. 2 and 3. The ultraviolet light irradiation method performs:
injecting transmission infrared rays of two polarized waves orthogonal to each other on the optical fiber 50 (step S11), converting the transmission infrared light propagated through the optical fiber 50 into ultraviolet light (step S12), and irradiating the ultraviolet light to a desired location Ar (step S13).

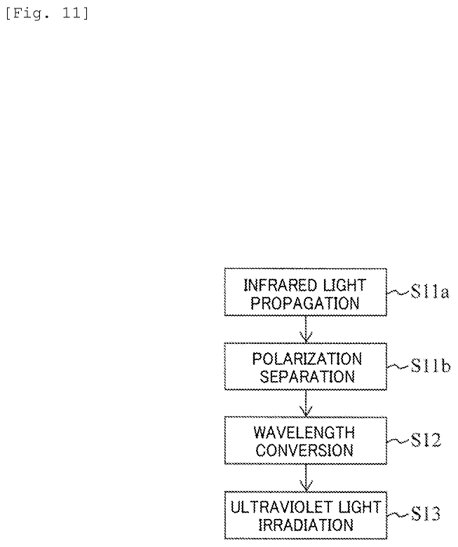

FIG. 11 is a flow chart for explaining an ultraviolet light irradiation method in an ultraviolet light irradiation system having the wavelength conversion unit 12 shown in FIGS. 4 and 5. The ultraviolet light irradiation method performs: injecting the transmission infrared light on the optical fiber 50 (step S11a),
separating the transmission infrared light propagated through the optical fiber 50 into orthogonal polarized waves (step S11b),
converting each of the separated transmission infrared light into ultraviolet light (step S12), and
irradiating the ultraviolet light to a desired location (step S13).

### REFERENCE SIGNS LIST

11 Light source unit
11a Infrared light source
11b Polarization scrambler
11c Polarization multiplexer
12 Wavelength conversion unit
12a Polarization separator
12b Polarization adjuster
12c Wavelength conversion device
13 Irradiation unit
14 Infrared light distribution unit
15 Ultraviolet light distribution unit
50 Optical fiber
51 Optical fiber
301 to 304 Ultraviolet light irradiation system

1. An ultraviolet light irradiation system comprising:
a light source unit that injects transmission infrared light of two polarized waves orthogonal to each other into an optical fiber,
a wavelength conversion unit that converts the transmission infrared light propagated through the optical fiber into ultraviolet light, and
an irradiation unit that irradiates the ultraviolet light to a desired location.

2. The ultraviolet light irradiation system according to claim 1, wherein
the light source unit includes a polarization scrambler, and the infrared light output from one infrared light source is used as the transmission infrared light by the polarization scrambler.

3. The ultraviolet light irradiation system according to claim 1, wherein
the light source unit includes a polarization multiplexer, and the transmission infrared light is obtained by multiplexing infrared light output from two infrared light sources having different output wavelengths by the polarization multiplexer.

4. An ultraviolet light irradiation system comprising:
a light source unit that injects transmission infrared light into an optical fiber, a wavelength conversion unit that converts the transmission infrared light propagated through the optical fiber into ultraviolet light, and
an irradiation unit that irradiates the ultraviolet light to a desired location; and
wherein the wavelength conversion unit has a polarization diversity configuration that separates the transmission infrared light into orthogonal polarized waves and converts each of the transmission infrared light into ultraviolet light.

5. The ultraviolet light irradiation system according to claim 1, wherein
the wavelength conversion unit supplies the ultraviolet light directly or with optical fibers to the irradiation unit.

6. The ultraviolet light irradiation system according to claim 1, wherein
at least one of the optical fiber that propagates the transmission infrared light and the optical fiber that supplies the ultraviolet light from the wavelength conversion unit to the irradiation unit is,
any one of a solid core optical fiber, a hole-assisted optical fiber, a hole-structured optical fiber, a hollow core optical fiber, a coupled core type optical fiber, a solid core type multi-core optical fiber, a hole-assisted type multi-core optical fiber, a hole-structured type multi-core optical fiber, a hollow core type multi-core optical fiber, and a coupled core type multi-core optical fiber.

7. An ultraviolet light irradiation method which performs:
injecting transmission infrared light of two polarized waves orthogonal to each other into an optical fiber,
converting the transmission infrared light propagated through the optical fiber into ultraviolet light, and
irradiating the ultraviolet light to a desired location.

8. (canceled)
